(19) United States
(12) Patent Application Publication
(10) Pub. No.: US 2010/0324130 A1
(43) Pub. Date: Dec. 23, 2010

(54) DAA PERIPHERAL BENZODIAZEPINE RECEPTOR LIGAND FOR CANCER IMAGING AND TREATMENT

(76) Inventors: Darryl J. Bornhop, Nashville, TN (US); Mingfeng Bai, Nashville, TN (US)

Correspondence Address: STITES & HARBISON PLLC, 401 COMMERCE STREET, SUITE 800, NASHVILLE, TN, 37219, US

(21) Appl. No.: 12/518,067

(22) PCT Filed: Dec. 5, 2007
(86) PCT No.: PCT/US2007/086538

§ 371 Date: Jul. 22, 2009

Related U.S. Application Data
(60) Provisional application No. 60/868,697, filed on Dec. 5, 2006.

(51) Int. Cl. A61K 31/335; C07C 233/00; C07D 403/06; C07C 237/00; C07D 493/00; G01N 21/00; A61K 31/16; A61P 35/00

(52) U.S. Cl. 514/452; 564/123; 548/490; 564/157; 549/364; 436/164; 514/616

(57) ABSTRACT

Peripheral Benzodiazepine Receptor (PBR) is an attractive target for tumor imaging and treatment due to its up-regulation in numerous cancer cell types. DAA1 106 is a selective PBR ligand with high binding affinity. Aspects of the present invention are series of functionalized DAA1 106 analogs, which can be conjugated to a variety of signaling and treatment moieties, and are widely applicable in PBR targeted molecular imaging and drug delivery.

### GOVERNMENT SUPPORT

This invention was made with Government support under Department of Defense Grant number W81XWH-04-1-0432. The Government has certain rights in this invention.

### BACKGROUND OF THE INVENTION

The Peripheral Benzodiazepine Receptor (PBR), an 18 kDa mitochondrial protein, has become an attractive target for cancer imaging and treatment.

Over-expression of PBR has been observed in a variety of cancers, including brain, breast, colorectal, prostate and ovary cancers, Hepatocellular carcinoma, astrocytomas and endometrial carcinoma, PBR is associated with a number of biological processes, such as cell proliferation, apoptosis, steroidogenesis, and immunomodulation, however, its exact physiological role is still not clear.

Several PBR-selective ligands have been discovered, including the diazepam derivative (Ro5-4864), the isoquinoline derivative (PK11195), the 2-acryl-3-indoleacetamide derivative (FGIN-1), and the phenoxyphenyl-acetamide derivative (DAA1106).

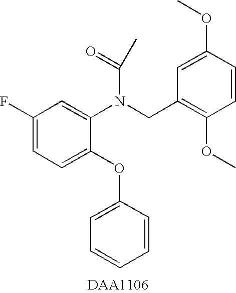

DAA1106 is an attractive PBR ligand because it has high binding affinity for PBR. Additionally, DAA1106 has been shown to displace PBR complexed PK11195 and Ro5-4864 at very low concentration (10⁻¹⁵-10⁻¹² M), however, 0.1-1 μM amounts of PK11195, Ro5-4864 or FGIN1 were necessary to displace DAA1106.

A conjugable form of DAA1106, which can be coupled to a variety of moieties, including signaling, therapeutic, and combinations thereof, is needed.

### SUMMARY OF THE INVENTION

An aspect of the present invention is conjugable DAA1106 compounds.

Another aspect of the present invention is a novel conjugate comprising a conjugable DAA1106 compound.

Another aspect of the present invention is imaging a molecular event comprising administering a conjugate of the present invention.

Another aspect of the present invention is a method of treating cancer comprising administering a conjugate of the present invention.

Another aspect of the present invention is a method of synthesizing receptor or protein targeted agents for selective cancer therapy. The methods of the present invention are designed to be applicable to the application of targeted delivery of any conjugable moiety (therapeutic, imaging or combination). Preparation of small molecule ligand that can be coupled to a drug, would allow the drug be selectively delivered and internalized into cells substantially improving cell killing and clinical efficacy.

In embodiments of the present invention, conjugable DAA1106 is the Peripheral Benzodiazepine Receptor (PBR) ligand.

Etoposide is one of the most widely used anticancer drugs and is active against small-cell lung cancers, leukemias, and lymphomas.

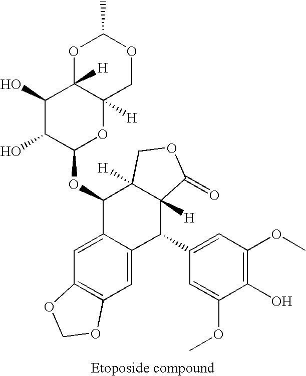

However, the application of etoposide in cancer therapy is limited by the lack of selectivity. PBR is a mitochondrial protein and highly expressed in leukemia and lymphoma cells. DAA1106 is a relatively new PBR ligand with fentomolar (10⁻¹⁵M) binding affinity for PBR. An embodiment of the present invention is coupling etoposide and other cancer therapeutics to DAA1106, and the resulting molecules can provide selective cancer therapy.

A conjugable form of DAA1106 with a carbon spacer (CnDAA1106, n=3-9) was therefore synthesized and used to conjugate etoposide. The compound CnDAA1106 is another embodiment of the present invention.

The present inventors have synthesized a functionalized PBR ligand, CₙDAA1106, which can be conjugated to a variety of signaling moieties and widely applied in PBR targeted cancer imaging and targeted drug delivery. In addition, these DAA1106 analogs of the present invention have been labeled with two fluorescent dyes and the resulting imaging probes, NIRDAA and LissDAA display nanomolar binding affinities to PBR and have been successfully imaged in vitro.

### BRIEF DESCRIPTION OF THE DRAWINGS

FIG. 1 is a chromatograph for NIR dye and NIRDAA at 780 nm.

FIG. 2 shows spectroscopy curves for LissDAA of the present invention.

FIGS. 3 and 4 are fluorescence microscopy, images showing cell uptake of NIRDAA and LissDAA.

### DESCRIPTION OF THE INVENTION

An embodiment of the present invention is a compound of the following formula:

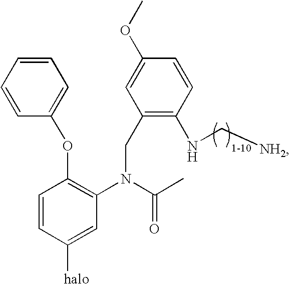

and stereoisomers and conjugable analogs thereof.

As indicated above, a conjugable analog of DAA1106 has been synthesized and characterized. The analog has a terminal amino group, facilitating coupling reactions.

The following is an example of a compound of the present invention:

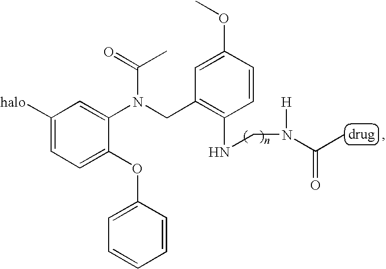

and stereoisomers and conjugable analogs thereof.

Additionally, the following is an example of a compound of the present invention:

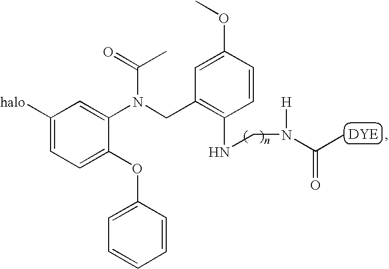

and stereoisomers and conjugable analogs thereof.

Unless disclosed otherwise, in the above examples and the compounds disclosed herein, N is an integer from 1 to 10.

Aspects of the invention related to imaging are carried out as described in Bornhop et al., US Application Publication Number 20060147379, incorporated herein by reference.

Two fluorescent dyes, IRDye™800CW NHS ester (LI-COR Biosciences, ε=300,000 L/mol cm in methanol) and lissamine™ rhodamine B sulphonyl chloride (Invitrogen, ε=300,000 L/mol cm in methanol) are examples of signaling parts to conjugate to the DAA1106 analog. The resulting compounds of the present invention have nanomolar binding affinities to PBR and appear to target PBR in vitro.

Also includes are dyes, such as, for example, near-infrared fluorophores/fluorescent dyes. Examples include cyanine dyes which have been used to label various biomolecules. See U.S. Pat. No. 5,268,486, which discloses fluorescent arylsulfonated cyanine dyes having large extinction coefficients and quantum yields for the purpose of detection and quantification of labeled components.

Additional examples include compounds of the following formulas:

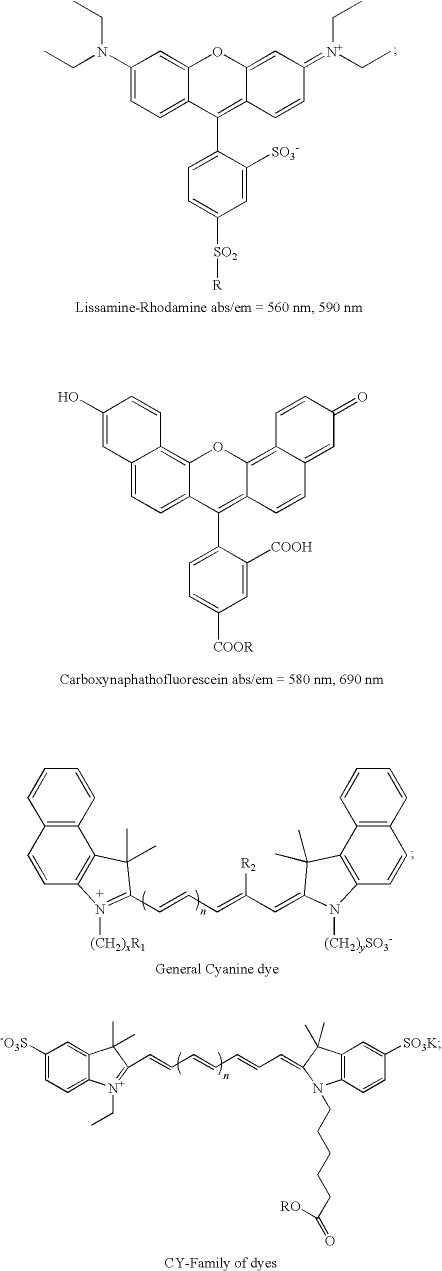

and analogs thereof.

Additional examples include dyes available from Li—Cor, such as IRDye™800CW,. For example, dyes disclosed in WO 02/24815, incorporated herein by reference, are dyes of the present invention. Furthermore, dyes disclosed in U.S. patent application Ser. No. 11/267,643, incorporated herein by reference, are dyes of the present invention. Additionally, dyes of U.S. Pat. No. 6,995,274, incorporated herein by reference, are dyes of the present invention.

Thus, a compound of the present invention is the following:

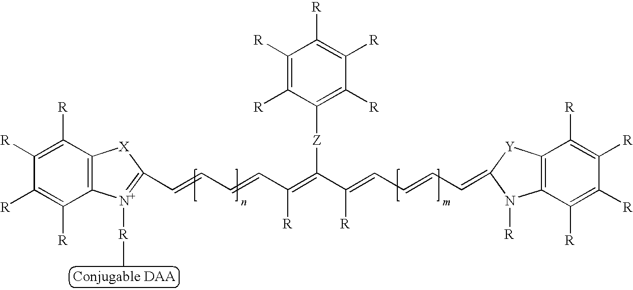

wherein the variables are defined in US patent application publication number 20060063247, incorporated herein by reference.

Additional examples of dyes usable with the present invention include dyes disclosed in U.S. Pat. No. 6,027,709. US '709 discloses dyes which have the following general formula:

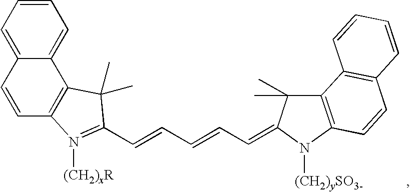

wherein R is —OH, —CO₂H, —NH₂, or —NCS and each of x and y, independently, is an integer selected from 1 to about 10. In preferred embodiments, each of x and y, independently, is an integer between about 2 and 6.

In one embodiment, the dye is N-(6-hydroxyhexyl)N′-(4-sulfonatobutyl)-3,3,3′,3′-tetramethylbenz(e)indodicarbocyanine, which has the formula:

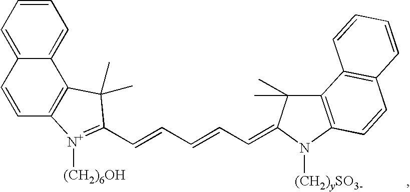

In a second embodiment, the dye is N-(5-carboxypentyl)N′-(4-sulfonatobutyl)3,3,3′,3′-tetramethylbenz(e)indodicarbocyanine, which has the formula:

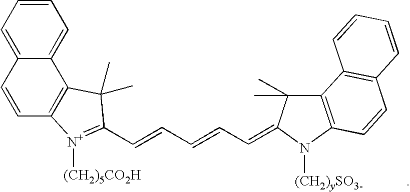

These two dyes are embodiments because they have commercially available precursors for the linking groups: 6-bromohexanol, 6-bromohexanoic acid and 1,4-butane sultone (all available from Aldrich Chemical Co., Milwaukee, Wis.). The linking groups provide adequate distance between the dye and the biomolecule for efficient attachment without imparting excessive hydrophobicity. The resulting labeled biomolecules retain their solubility in water and are well-accepted by enzymes.

These dyes, wherein R is —CO₂ H or —OH can be synthesized, as set forth in detail in the US '709 patent, by reacting the appropriate N-(carboxyalkyl)- or N-(hydroxyalkyl)-1,1,2-trimethyl-1H-benz(e)indolinium halide, preferably bromide, with sulfonatobutyl-1,1,2-trimethyl-1H-benz(e)indole at a relative molar ratio, of about 0.9:1 to about 1:0.9, preferably 1:1 in an organic solvent, such as pyridine, and heated to reflux, followed by the addition of 1,3,3-trimethoxypropene in a relative molar ratio of about 1:1 to about 3:1 to the reaction product and continued reflux. The mixture subsequently is cooled and poured into an organic solvent such as ether. The resulting solid or semi-solid can be purified by chromatography on a silica gel column using a series of methanol/chloroform solvents.

As an alternative, two-step, synthesis procedure, also detailed in U.S. '709, N-4-sulfonatobutyl-1,1,2-trimethyl-1H-benz(e)indole and malonaldehyde bis(phenylimine)-monohydrochloride in a 1:1 molar ratio can be dissolved in acetic anhydride and the mixture is heated. The acetic anhydride is removed under high vacuum and the residue is washed with an organic solvent such as ether. The residual solid obtained is dried and subsequently mixed with the appropriate N-(carboxyalkyl)- or N-(hydroxyalkyl)-1,1,2-trimethyl-1H-benz(e)indolinium halide in the presence of an organic solvent, such as pyridine. The reaction mixture is heated, then the solvent is removed under vacuum, leaving the crude desired dye compound. The procedure was adapted from the two step procedure set forth in Ernst, L. A., et al., Cytometry 10:3-10 (1989).

The dyes also can be prepared with an amine or isothiocyanate terminating group. For example, N-(omega-amino-alkyl)-1,1,2-trimethyl-1H-benz.(e)indolenium bromide hydrobromide (synthesized as in N. Narayanan and G. Patonay, J. Org. Chem. 60:2391-5 (1995)) can be reacted to form dyes of formula I wherein R is —NH₂. Salts of these amino dyes can be converted to the corresponding isothiocyanates by treatment at room temperature with thiophosgene in an organic solvent such as chloroform and aqueous sodium carbonate.

These dyes have a maximum light absorption which occurs near 680 nm. They thus can be excited efficiently by commercially available laser diodes that are compact, reliable and inexpensive and emit light at this wavelength. Suitable commercially available lasers include, for example, Toshiba TOLD9225, TOLD9140 and TOLD9150, Phillips CQL806D, Blue Sky Research PS 015-00 and NEC NDL 3230SU. This near infrared/far red wavelength also is advantageous in that the background fluorescence in this region normally is low in biological systems and high sensitivity can be achieved.

The hydroxyl, carboxyl and isothiocyanate groups, of the dyes provide linking groups for attachment to a wide variety of biologically important molecules, including proteins, peptides, enzyme substrates, hormones, antibodies, antigens, haptens, avidin, streptavidin, carbohydrates, oligosaccharides, polysaccharides, nucleic acids, deoxy nucleic acids, fragments of DNA or RNA, cells and synthetic combinations of biological fragments such as peptide nucleic acids (PNAs).

In another embodiment of the present invention, the ligands of the present invention may be conjugated to a lissamine dye, such as lissamine rhodamine B sulfonyl chloride. For example, a conjugable form of DAA1106 may be conjugated with lissamine rhodamine B sulfonyl chloride to form a compound of the present invention.

Lissamine dyes are typically inexpensive dyes with attractive spectral properties. For example, lissamine rhodamine B sulfonyl chloride has a molar extinction coefficient of 88,000 cm⁻¹M⁻¹ and good quantum efficient of about 95%. It absorbs at about 568 nm and emits at about 583 nm (in methanol) with a decent stokes shift and thus bright fluorescence.

Coupling procedures for the PBR ligands proceed via standard methods and will be recognized by those skilled in the art. In general, the nucleophilic N terminuses of the targeting moieties are reactive towards activated carbonyls, for example an NHS (N-hydroxysuccinimide ester), sulfonyl chlorides, or other electrophile bearing species. Solvent of choice for coupling reactions can be dye specific, but include dimethyl sulfoxide (DMSO), chloroform, and/or phosphate buffered saline (PBS buffer). The resulting conjugates, amides, sulfonamides, etc. resist hydrolysis under physiological conditions, and are thus useful for in-vivo and in-vitro application.

The administration step may be in vivo administration or in vitro administration. The in vivo administration step further comprises at least one time course imaging determination, and in other embodiments, the in vivo administration step further comprises at least one bio distribution determination.

As an example of a conjugable DAA1106 synthetic pathway is shown in Scheme 1. Compound 1 was synthesized as previously reported. The alkylation reaction of 1 with 2-bromo-5-methoxybenzyl bromide was straightforward and produced 2 in 99% yield. Aromatic substitution of a diamine (with 3-9 carbon linker) resulted in relatively low yield (6%-33%). This is due to several byproducts and decomposition of desired product prior to reaction completion. The optimal reaction time for the conjugable DAA1106 reaction is listed in Table 1.

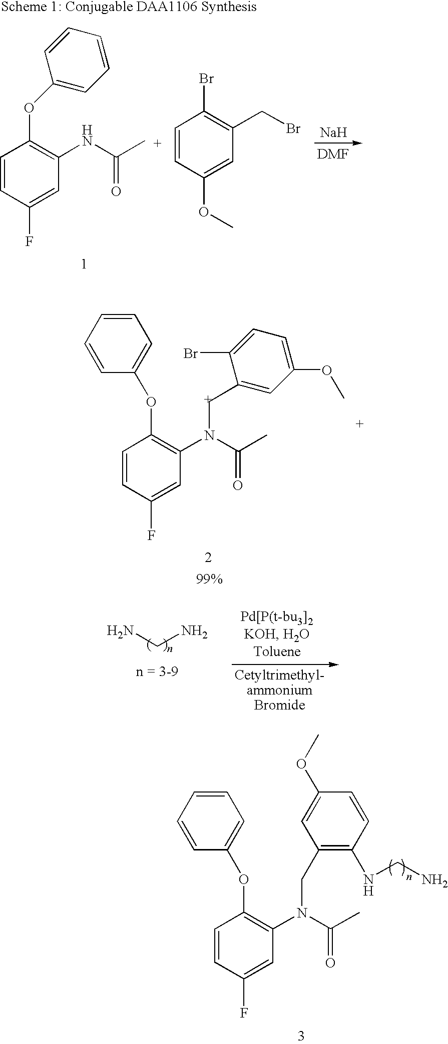

TABLE 1
CₙDAA1106 reactions summary

| CₙDAA1106 | Reaction time (hr) | Yield (%) |
|---|---|---|
| C₃DAA1106 | 3 | 8.7 |
| C₄DAA1106 | 2 | 7.9 |
| C₅DAA1106 | 2 | 10 |
| C₆DAA1106 | 6 | 33 |
| C₇DAA1106 | 2.5 | 12 |
| C₈DAA1106 | 2.5 | 5.8 |
| C₉DAA1106 | 2.5 | 11 |

The effect of spacer length on the binding affinity of the conjugable DAA1106 analog has also been investigated. More specifically, conjugable form of DAA1106, with 3-9 carbon spacers, has been synthesized, characterized, and used in a competitive binding assay. The amino group was capped by acetyl group to reduce non-specific binding (Scheme 2). The binding affinity data is shown in Table 2. Conjugable DAA1106 with a 3 carbon linker (C₃DAA1106) (IC50=0.39 μM) and C7DAA1106 (IC50=0.40 μM) have higher binding affinities than C₄DAA1106 (IC50=0.80 μM) and C₅DAA1106 (IC50=0.84 μM), but relatively low binding affinities compared to C₆DAA1106 (IC50=0.29 μM), C₈DAA1106 (IC50=0.24 μM) and C9DAA1106 (IC50=0.29 μM). Even though these binding affinities are much lower than DAA1106 (IC50=0.28 nM) and [¹¹C]DAA1106 (IC50=0.91 nM), the nanomolar binding affities (Kᵢ=43-149 nM) appear rather promising. A six carbon linker seems to be the most optimal due to relatively high binding affinity and yield (33%) of C6DAA1106.

TABLE 2
Capped CₙDAA1106 binding studies

| CₙDAA1106 | IC50 (μM) | Ki (nM) |
|---|---|---|
| C₃DAA1106 | 0.39 ± 0.13 | 68 ± 23 |
| C₄DAA1106 | 0.80 ± 0.21 | 141 ± 36 |
| C₅DAA1106 | 0.84 ± 0.28 | 149 ± 49 |
| C₆DAA1106 | 0.35 ± 0.22 | 52 ± 30 |
| C₇DAA1106 | 0.40 ± 0.19 | 71 ± 33 |
| C₈DAA1106 | 0.24 ± 0.11 | 43 ± 19 |
| C₉DAA1106 | 0.29 ± 0.09 | 51 ± 17 |

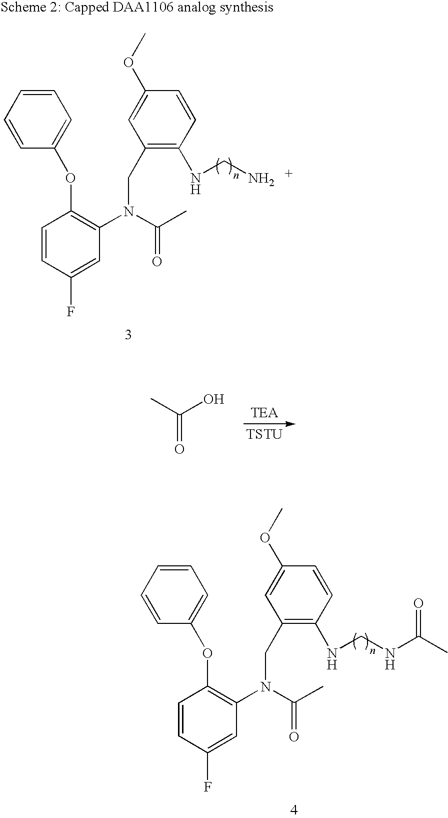

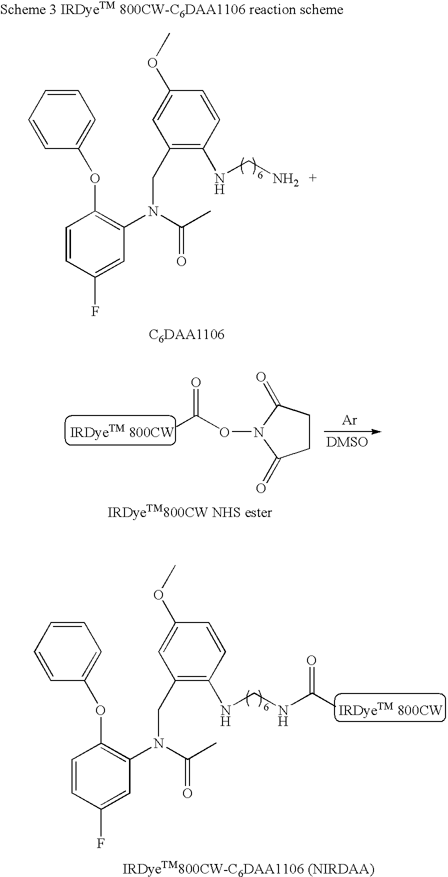

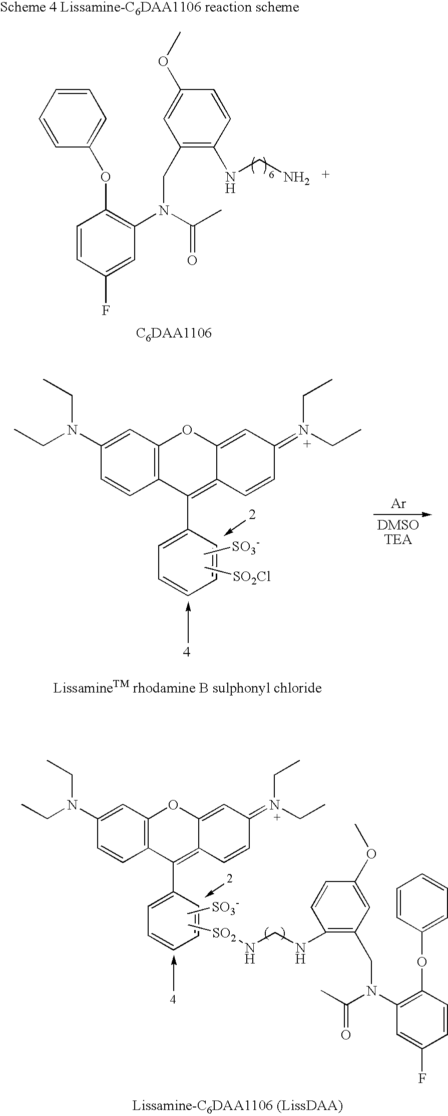

Since NIR probes capable of targeting specific receptors appear to be powerful noninvasive imaging tools for preclinical diagnosis, we conjugated our relative high binding affinity PBR targeted ligand, C₆DAA1106, to IRDye™800CW NHS ester. The reaction was straightforward, but the overall yield was relatively low (31%), mainly due the impurities in the dye sample and side reactions.

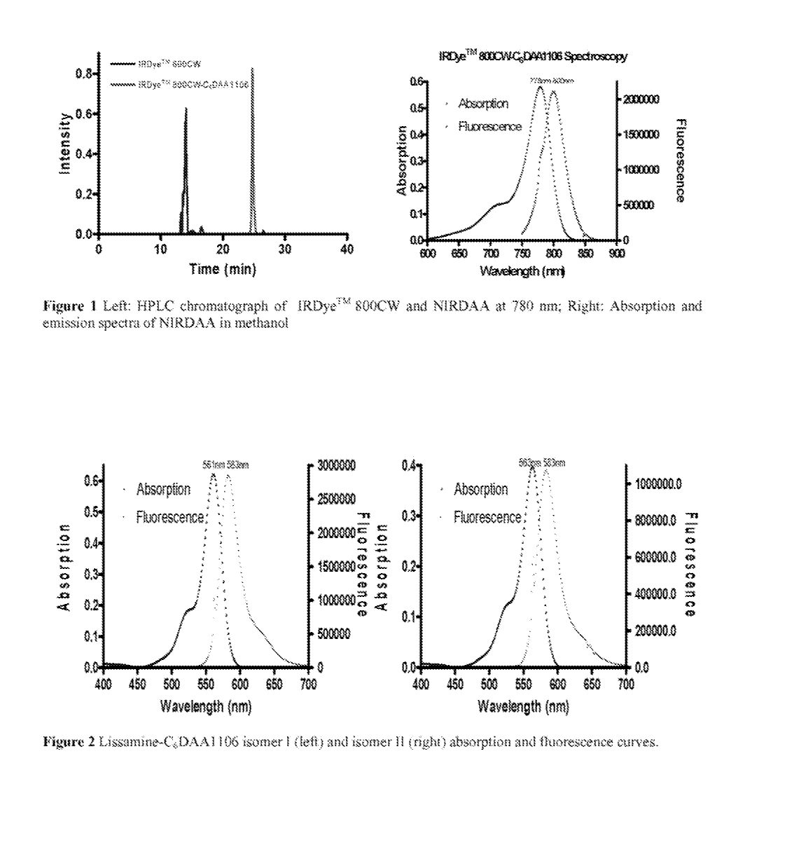

HPLC was used to monitor the production of IRDye™800CW-C₆DAA1106 (NIRDAA). The chromatographs for both the NIR dye and NIRDAA at 780 nm are shown in FIG. 1. FIG. 1 shows the excitation and emission spectra. The excitation of NIRDAA at 778 nm and its subsequent NIR emission at 800 nm allows deep tissue penetration with reduced absorption and scattering for in vivo imaging.

Lissamine™ rhodamine B sulphonyl chloride was also used to conjugate C₆DAA1106. Even though not a NIR dye, the lissamine dye is optimized for commonly used texas red filter set and well known for providing high quality images. Since the commercially available lissamine dye has two isomers, the conjugation reaction yielded two isomers as well. The spectroscopy curves are shown in FIG. 2. Isomer I, which has higher molar extinction coefficient (ε=124,000 L/mol cm in methanol) than isomer II (ε=80,000 L/mol cm in methanol), was selected for imaging.

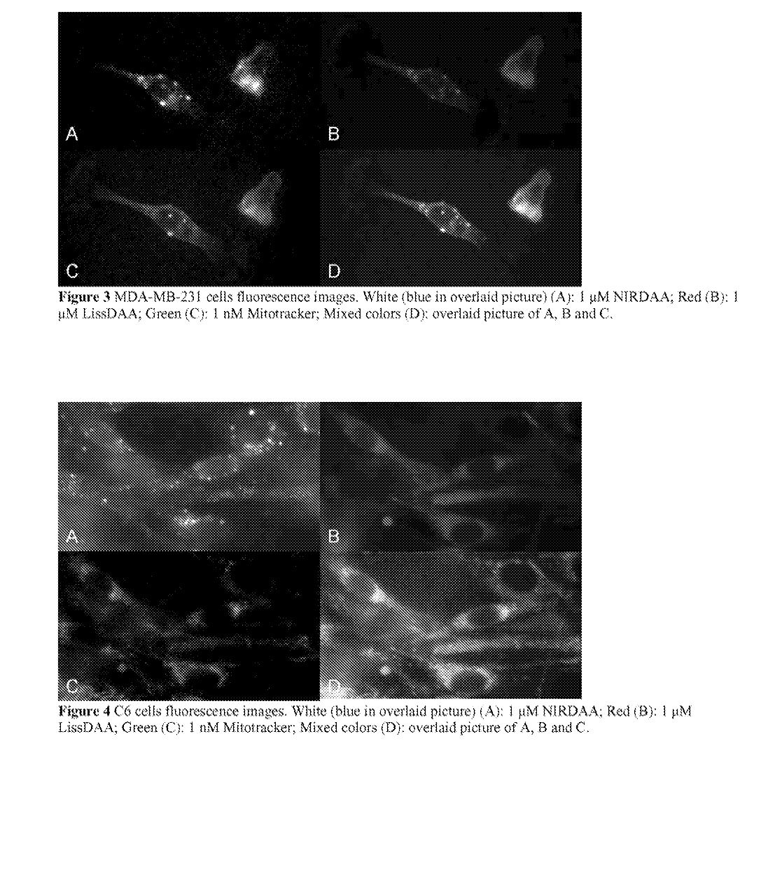

Fluorescence microscopy imaging studies were performed to investigate the cell uptake of NIRDAA and LissDAA in MDA-MB-231 (human metastic mammary adenocarcinoma) and C6 (rat glioma) cells. Accumulation of both agents in these cells was found (FIGS. 3 & 4). In addition, MitoTracker Green, which labels mitochondria proteins, was co-incubated with these two molecules in cells. Overlaid pictures demonstrate co-localization of all three molecules, which suggests that the optical probes selectively bind PBR. The nanomolar binding affinities (Kᵢ=42 nM for NIRDAA and 0.91 nM for LissDAA) provided further evidence on the selective binding.

As stated above, embodiments of the present invention is a compound of the present invention:

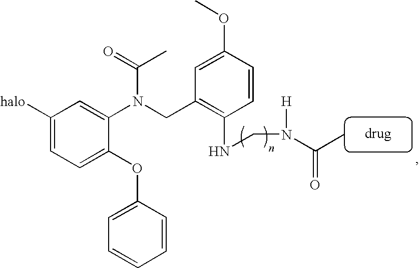

and stereoisomers and conjugable analogs thereof.

In embodiments of the present invention, a chemotherapeutic agent is the “drug.” An embodiment of the chemotherapeutic agent is a topoisomerase inhibitor. A topoisomerase inhibitor may be adriamycin, amsacrine, camptothecin, daunorubicin, dactinomycin, doxorubicin, eniposide, epirubicin, etoposide, idarubicin, mitoxantrone, teniposide, or topotecan. Preferably, the topoisomerase inhibitor is etoposide.

The imaging and/or therapeutic agents of the present invention may be administered as determined by one of ordinary skill in the art. In embodiments the agents may be administered as shown in U.S. application Ser. No. 11/181201, incorporated herein by reference.

That is, compounds of the present invention can be administered orally, parenterally by intravenous injection, transdermally, by pulmonary inhalation, by intravaginal or intrarectal insertion, by subcutaneous implantation, intramuscular injection or by injection directly into an affected tissue, as for example by injection into a tumor site. In some instances the materials may be applied topically at the time surgery is carried out. In another instance the topical administration may be ophthalmic, with direct application of the therapeutic composition to the eye.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; For oral dosage, a syrup, tablet or palatable solution; for topical application, a lotion, cream, spray or ointment; for administration by inhalation, a microcrystalline powder or a solution suitable for nebulization; for intravaginal or intrarectal administration, pessaries, suppositories, creams or foams. Preferably, the route of administration is parenteral, more preferably intravenous.

In general, an embodiment of the invention is to administer a suitable daily dose of a therapeutic composition that will be the lowest effective dose to produce a therapeutic effect. However, it is understood by one skilled in the art that the dose of the composition to practice the invention will vary depending on the subject and upon the particular route of administration used. It is routine in the art to adjust the dosage to suit the individual subjects. Additionally, the effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The actual dosage suitable for a subject can easily be determined as a routine practice by one skilled in the art, for example a physician or a veterinarian given a general starting point.

The therapeutic treatment may be administered hourly, daily, weekly, monthly, yearly (e.g., in a time release form) or as a one-time delivery. The delivery may be continuous delivery for a period of time, e.g., intravenous delivery. In one embodiment of the methods described herein, the therapeutic composition is administered at least once per day. In one embodiment, the therapeutic composition is administered daily. In one embodiment, the therapeutic composition is administered every other day. In one embodiment, the therapeutic composition is administered every 6 to 8 days. In one embodiment, the therapeutic composition is administered weekly.

In embodiments of the methods described herein, the route of administration can be oral, intraperitoneal, transdermal, subcutaneous, by vascular injection into the tumor, by intravenous or intramuscular injection, by inhalation, topical, intralesional, infusion; liposome-mediated delivery; intrathecal, gingival pocket, rectal, intrabronchial, nasal, transmucosal, intestinal, ocular or otic delivery, or any other methods known in the art as one skilled in the art may easily perceive. In other embodiments of the invention, the compositions incorporate particulate forms protective coatings, hydrolase inhibitors or permeation enhancers for various routes of administration, including parenteral, pulmonary, nasal and oral.

An embodiment or the method of present invention is to administer the compositions described herein in a sustained release form. Such method comprises implanting a sustained-release capsule or a coated implantable medical device so that a therapeutically effective dose is continuously delivered to a subject of such a method. The compositions may be delivered via a capsule which allows sustained-release of the agent or the peptide over a period of time. Controlled or sustained-release compositions include formulation in lipophilic depots (e.g., fatty acids, waxes, oils). Also comprehended by the invention are particulate compositions coated with polymers (e.g., poloxamers or poloxamines).

The method of present invention is effective in treatment of various types of cancers, including but not limited to: pancreatic cancer, renal cell cancer, Kaposi's sarcoma, chronic leukemia (preferably chronic myelogenous leukemia), chronic lymphocytic leukemia, breast cancer, sarcoma, ovarian carcinoma, rectal cancer, throat cancer, melanoma, colon cancer, bladder cancer, lymphoma, mesothelioma, mastocytoma, lung cancer, liver cancer, mammary adenocarcinoma, pharyngeal squamous cell carcinoma, gastrointestinal cancer, stomach cancer, myeloma, prostate cancer, B-cell malignancies or metastatic cancers.

The present invention is also effective against other diseases related to unwanted cell proliferation. Such hyperproliferative diseases include but are not limited to: psoriasis, rheumatoid arthritis, lamellar ichthyosis, epidermolytic hyperkeratosis, restenosis, endometriosis, proliferative retinopathy, lung fibrosis, desmoids or abnormal wound healing.

### Experimental Examples

The following examples are presented to show various embodiments of the present invention, and should be interpreted as such. They are not to be construed as being limiting of, or defining the boundaries of, the present invention.

A synthetic pathway of etoposide-C6DAA1106 is shown below:

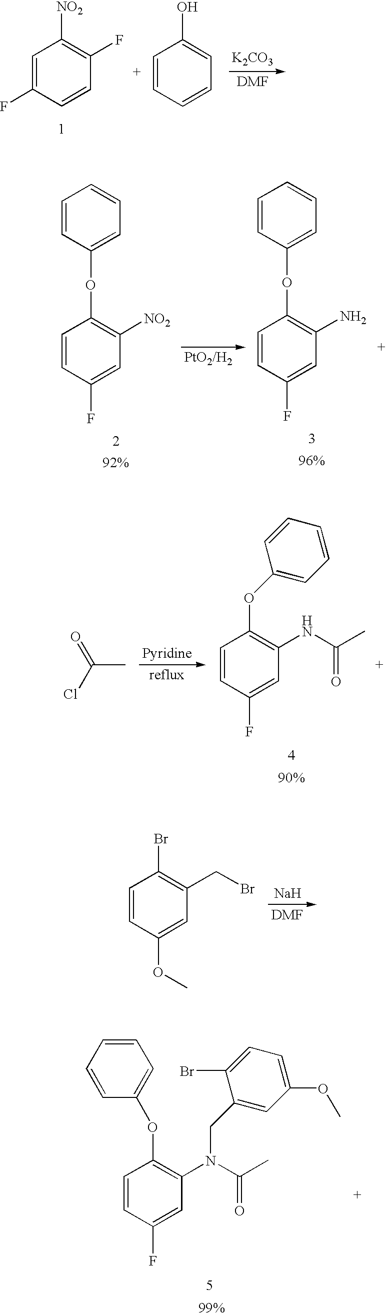

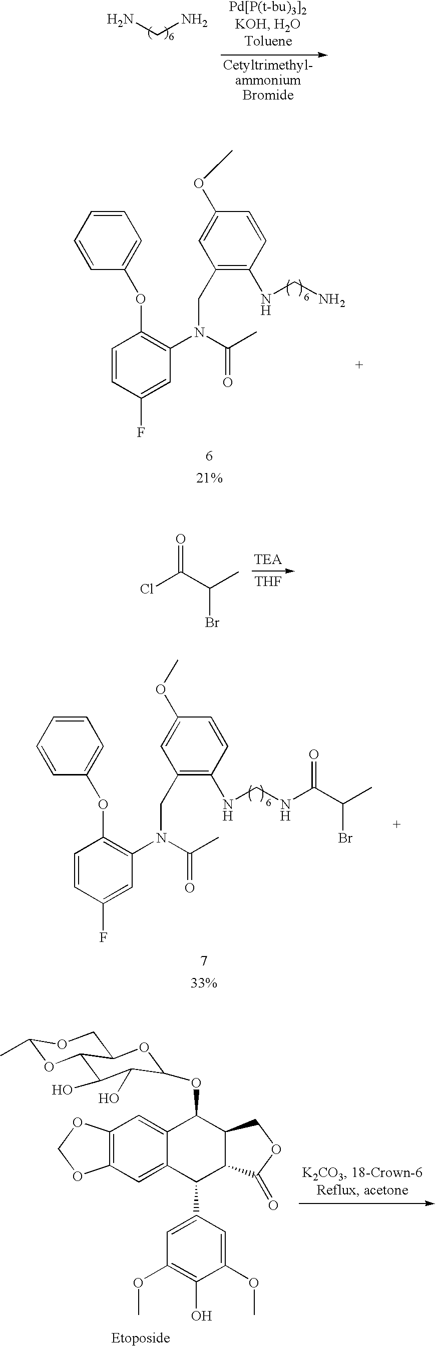

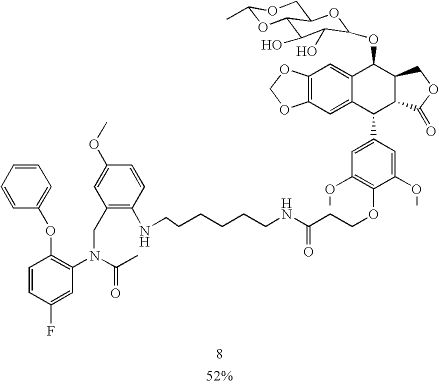

### 5-fluoro-2-phenoxynitrobenzene 2

A mixture of 2,5-difluoronitrobenzene 1 (11.6 g, 73 mmol), phenol (7.2 g, 76 mmol) and K2CO3 (11.1 g, 80 mmol) in DMF (40 mL) was stirred at 75° C. for 10 hours: The mixture was concentrated by vacuum, and the residue was partitioned between ethyl acetate and water. The separated organic phase was washed with 1 M aqueous NaOH, 1 M aqueous HCl, saturated aqueous NaHCO3 and then saturated brine, dried over MgSO4, filtered and evaporated to dryness. The residue was chromatographed (silica gel) using 15:1 hexanes/AcOEt as the elutent to obtain 5-fluoro-2-phenoxynitrobenzene 2 as yellow oil (15.7 g, 92%): 1H-NMR (300 MHz, CDCl3) δ 6.91-7.50 (m, 7H), 7.71(dd, J=7.7, 3.1 Hz, 1H); MS (GC) m/z 233(M, 100%).

### 5-fluoro-2-phenoxyaniline 3

A mixture of 5-fluoro-2-phenoxynitrobenzene 2 (15.2 g, 65 mmol) and PtO2 (131 mg) in MeOH (65 mL) was stirred at 50° C. for 5 hours under a hydrogen atmosphere. The mixture was filtered through celite. The filtrate was evaporated to dryness to yield 5-fluoro-2-phenoxyaniline 3 as brown oil (12.7 g, 97%): 1H-NMR (300 MHz, CDCl3) δ 3.88 (br s, 2H), 6.40 (ddd, J=8.6, 8.6, 3.1 Hz, 1H), 6.53(dd, J=9.9, 3.1 Hz, 1H), 6.84 (dd, J=8.6, 5.5 Hz, 1H), 6.86-7.13 (m, 3H), 7.21-7.40 (m, 2H); MS (GC) m/z 203(M, 100%). The product was used in the next step without further purification.

### N-(5-fluoro-2-phenoxyphenyl)acetamide 4

5-fluoro-2-phenoxyaniline 3 (5.14 g, 25 mmol) was dissolved in pyridine (15 mL) in a dry flask. At 0° C., acetyl chloride (2.3 mL, 33 mmol) was slowly added to the reaction, which was then refluxed for one hour, and subsequently, concentrated by vacuum. The residue was purified via column chromatography (silica gel) using CHCl3 as the eluent to give N-(5-fluoro-2-phenoxyphenyl)acetamide 4 as white solid(5.6 g, 90%): 1H-NMR (300MHz., CDCI3) δ 8.29 (dd, J=10.5, 3.0 Hz, 1H), 7.74 (br s, 1H),7.36 (t, J=7.5 Hz, 2H), 7.15 (t, J=7.5 Hz, 1H), 6.98 (d, J=7.5 Hz, 2H), 6.81 (2d, J=5.4, 5.1 Hz, 1H), 6.70 (2dd, J1=J2=J3=J4=3 Hz, 1H), 2.17 (s, 3H). MS (GC) m/z 245(M, 100%)

### N-(2-bromo-5-methoxybenzyl)-N-(5-fluro-2-phenoxphenyl)acetamide

To a dry round bottom flask, was added dry DMF (10 mL) and sodium hydride (100 mg), followed by N-(5-fluoro-2-phenoxyphenyl)acetamide 4 (1.08 g, 4.4 mmol). After the solution was stirred for 15 minutes, 2-bromo-5-methoxy-benzyl bromide (1.4 g, 5.0 mmol) was added. After 30 minutes, the reaction was added to stirring water chilled to 0° C. (60 mL). The mixture was extracted by dichloromethane three times. The organic solutions were combined, dried over Mg2SO4 and then evaporated to dryness. The residue was chromatographed (silica gel) using 1:3 Ethyl acetate/hexanes as the eluent to yield N-(2-bromo-5-methoxybenzyl)-N-(5-fluro-2-phenoxphenyl)acetamide 5 as yellow oil(1.94 g, 99%). 1H-NMR (300 MHz, CDCl3) δ 7.29-7.33 (m, 3H), 7.12 (t, J=7.6 Hz, 1H), 7.02 (d, J=3.2 Hz, 1H), 6.80-6.96 (m, 5H), 6.62 (dd, J=8.4, 2.8 Hz, 1H), 4.96 (dd, J=178, 15.2 Hz, 2H), 3.66 (s, 3H), 2.00 (s, 3H). MS (ESI)+m/z 442.8([MH]+, 100%), 444.8([MH]+, 100%).

C6DAA1106 6

N-(2-bromo-5-methoxybenzyl)-N-(5-fluro-2-phenoxphenyl)acetamide 5 (202 mg, 0.45 mmol), Pd[P(t-Bu)3]2 (4.6 mg, 9 μmol), hexamethylenediamine (158.4 mg, 1.36 mmol), potassium hydroxide (38.2 mg, 0.68 mmol), cetyltrimethylammonium bromide (1.6 mg, 4.4 μmol), water (12.2 μL, 0.68 mmol) and 800 μL dry toluene were placed in a round bottom flask flushed with argon. The flask was sealed with a septum and the reaction mixture was stirred vigorously at 90° C. for three hours. The reaction was then concentrated by vacuum and purified by column chromatography using 9:1:0.1 CH2Cl2/CH3OH/NH3.H2O to yield C6DAA1106 as colorless oil (45.4 mg, 21%), 1H-NMR (300 MHz, CDCl3) δ 7.28 (t, J=8.1 Hz, 2H), 7.10 (t, J=7.5 Hz, 1H), 6.91-6.95 (n, 1H), 6.79-6.84 (m, 1H), 6.70-6.75 (4H, m), 6.40 (d, J=8.7 Hz), 6.20 (1H, d, J=3 Hz, 1H), 4.76 (AX, J=14.7 Hz, ??=95.1 Hz, 2H), 3.61 (3H, s), 2.87-3.03 (2H, m, 2H), 2.69 (2H, J=6.9 Hz, 2H), 1.94 (3H), 1.54-1.61 (m, 2H), 1.28-0.48 (m, 8H). MS (ESI)⁺ m/z 480.2 ([MH]+, 100%).

### 2-bromo-N-(6-(2-((N-(5-fluoro-2-phenoxyphenyl)acetamido)methyl)-4-methoxyphenylamino)hexyl)propanamide 7

A solution of C6DAA11066 (48 mg, 0.1 mmol) and TEA (14.5 μL, 0.1 mmol) in dry THF (4 mL) was cooled by dry ice/acetone. 2-bromo-propionyl chloride (10.1 μL, 0.1 mmol) was then added and the resultant mixture was allowed to stir for 10 minutes. Triethyl ammonium chloride salt was filtered through filter paper. Solvent was removed by vacuum and the product was purified by silica gel column chromatography using methylene chloride/methanol 32/1 as eluent to give 2-bromo-N-(6-(2-((N-(5-fluoro-2-phenoxyphenyl)acetamido)methyl)-4-methoxyphenylamino)hexyl)propanamide 2 as colorless oil (20 mg, 33%). 1H NMR 300 MHz (CDCl3) δ 7.30-7.26 (m, 2H), 7.10 (t, J=7.5 Hz, 1H), 6.97-6.91 (m, 1H), 6.84-6.79 (m, 1H), 6.77-6.68 (m, 4H), 6.40 (d, J=8.7 Hz, 1H), 6.19 (d, J=2.7 Hz, 1H), 4.93-4.86 (m, 1H), 4.66-4.59 (m, 1H), 4.41 (q, J=10.5, 3.6 Hz, 1H), 3.60 (s, 3H), 3.27 (q, J=9.8, 3.2 Hz, 2H), 3.02-2.88 (m, 2H), 1.95 (s, 3H), 1.86 (d, J=6.9 Hz, 3H), 1.64-1.50 (m, 4H), 1.47-1.31 (m, 4H). MS (ESI) m/z 614.6 Da [M+H] (100%) 616.7 Da [M+H] (100%)

Etoposide-C6DAA1106 8

A mixture of etoposide (12 mg, 20 μmol), K2CO3 (4 mg, 30 μmol) and 18-crown-6 (1 mg, 4 μmol) in dry acetone (1 mL) was stirred for 5 minutes under room temperature. A solution of 7 (6 mg, 10 μmol) in dry acetone (300 μL) was added to the mixture and the reaction was heated to reflux with vigorous stirring. After 6 hours, the reaction was allowed to cool down to room temperature and the solvent was removed by vacuum. The residue was partitioned between water and dichloromethane. Extracted with dichloromethane three times. The organic layers were combined, dried over sodium sulfate and concentrated by vacuum. The residue was purified by silica gel column chromatography using 3% methanol in dichloromethane as eluent to give etoposide-C6DAA1106 8 as colorless oil (5.7 mg, 52%). 1H NMR 300 MHz (CDCl3) δ 7.81 (m, 1H), 7.28 (t, J=7.2 Hz, 1H), 7.10 (t, J=7.2 Hz, 1H), 6.95-6.92 (m, 1H), 6.83-6.69 (m, 6H), 6.45 (d, J=3.2 Hz, 2H), 6.41-6.38 (m, 2H), 6.19 (t, J=2.4 Hz, 1H), 5.96-5.94 (m, 2H), 4.94-4.85 (m, 3H), 4.73-4.47 (m, 5H), 4.25-4.23 (m, 1H), 4.18-4.14 (m. 1H), 3.93 (t. J=6.8 Hz, 1H), 3.81 (s, 6H), 3.60 (s, 3H), 3.58-3.56 (m, 2H), 3.44 (t, J=9.2 Hz, 1H), 3.35-3.26 (m, 3H), 3.18-3.13 (m, 2H), 3.00-2.89 (m, 4H), 1.94-1.92 (m, 3H), 1.62-1.52 (m, 11H), 1.42-1.35 (m, 8H). MS (ESI) m/z 1122.7 Da [M+H] (100%).

In other examples of the present invention, the following conjugable DAA 1106 compounds were made:

C₃DAA1106 (conjugable DAA1106 with three carbon linker)(Y=8.7%)

### C₄DAA1106 (Y=7.9%)

### C₅DAA1106 (Y=10%)

¹H-NMR (400 MHz, CDCl₃) δ 7.27 (t, J=8.4 Hz, 2H), 7.09 (t, J=7.6 Hz, 1H), 6.91-6.96 (m, 1H), 6.79-6.83 (m, 1H), 6.69-6.75 (m, 4H), 6.39 (d, J=8.8 Hz, 1H), 6.19 (d, J=2.8 Hz, 1H), 4.75 (AX, J=9.6 Hz, Δv=79.2 Hz, 2H), 3.60 (s, 3H), 2.86-3.03 (m, 2H), 2.77 (t, J=5.1 Hz, 2H), 1.94 (s, 3H), 1.50-1.62 (m, 4H), 1.40-1.45 (m, 2H). MS (ESI)⁺ m/z 466.3 ([MH]⁺, 100%)

### C₆DAA1106 (Y=33%)

¹H-NMR (300 MHz, CDCl₃) δ 7.28 (t, J=8.1 Hz, 2H), 7.10 (t, J=7.5 Hz, 1H), 6.91-6.95 (m, 1H), 6.79-6.84 (m, 1H), 6.70-6.75 (m, 4H), 6.40 (d, J=8.7 Hz, 1H), 6.20 (d, J=3 Hz, 1H), 4.76 (AX, J=14.7 Hz, Δv=95.1 Hz, 2H), 3.61 (s, 3H), 2.87-3.03 (m, 2H), 2.66 (t, J=6.9 Hz, 2H), 1.94 (s, 3H), 1.54-1.61 (m, 2H), 1.28-0.48 (m, 8H). MS (ESI)⁺ m/z 480.2 ([MH]⁺, 100%).

### C₇DAA1106 (Y=12%)

¹H-NMR (300 MHz, CDCl₃) δ 7.28 (t, J=7.5 Hz2H), 7.10 (t, J=7.5 Hz, 1H), 6.91-6.98 (m, 1H), 6.80-6.85 (m, 1H), 6.70-6.75 (m, 4H), 6.41 (d, J=9.0 Hz, 1H), 6.20 (d, J=3.0 Hz, 1H), 4.76 (AX, J=14.7 Hz, Δv=103.2 Hz, 2H), 3.60 (s, 3H), 2.84-3.06 (m, 2H), 2.68 (t, J=7.2 Hz, 2H), 1.94 (s, 3H), 1.53-1.60 (m, 2H), 1.31-1.46(m, 12H). (ESI)⁺ m/z 494.3 ([MH]⁺, 100%)

### C₈DAA1106 (Y=5.8%)

¹H-NMR (400 MHz, CDCl₃) δ 7.28 (t, J=8.4 Hz, 2H), 7.10 (t, J=7.6 Hz, 1H), 6.92-6.97 (m, 1H), 6.81-6.84 (m, 1H), 6.71-6.74 (m, 4H), 6.41 (d, J=8.8 Hz, 1H), 6.20 (d, J=2.8 Hz, 1H), 4.76 (AX, J=14.4 Hz, Δv=140.4 Hz, 2H), 3.60 (s, 3H), 2.85-3.04 (m, 2H), 2.72 (t, J=7.2 Hz, 2H), 1.94 (s, 3H), 1.44-1.59 (m, 6H), 1.30-1.37(m, 10H). (ESI)⁺ m/z 508.3 ([MH]⁺, 100%)

### C₉DAA1106 (Y=11%)

¹H-NMR (300 MHz, CDCl₃) δ 7.29 (t, J=7.5 Hz, 2H), 7.10 (t, J=7.5 Hz, 1H), 6.92-6.97 (m, 1H), 6.80-6.85 (m, 1H), 6.71-6.75 (m, 4H), 6.41 (d, J=9.0 Hz, 1H), 6.20 (d, J=3.0 Hz, 1H), 4.76 (AX, J=14.4 Hz, Δv=105.3 Hz, 2H), 3.61 (s, 3H), 2.84-3.06 (m, 2H), 2.72 (t, J=6.9 Hz, 2H), 2.51 (s, 2H), 1.94 (s. 3H), 1.45-1.62 (m, 5H), 1.19-1.36(m, 10H). (ESI) m/z 522.3 ([MH]⁺, 100%)

### General Method for Conjugable DAA1106 Amide (4,) Synthesis

A mixture of acetic acid (1.6 μL, 27.5 μmol), triethyl amine (TEA) (50 μL) and 2-Succinimido-1,1,3,3,-tetramethyluronium tetrafluoroborate (TSTU) (8.3 mg, 27.5 μmol) in dry methylene chloride (1 mL) was stirred at room temperature under argon for three hours. A solution of conjugable DAA1106 (25 μmol) in anhydrous methylene chloride (1 mL) was added to the mixture and the resulting mixture was stirred for another two and half hours. Reaction solution was then concentrated by vacuum rotary evaporation and the product was purified via silica gel column chromatography using 3% methanol in methylene chloride as eluent. Conjugable DAA1106 amide was collected as a colorless oil.

### C₃DAA1106 Amide

¹H-NMR (300 MHz, CDCl₃) δ 7.27 (t, J=7.5 Hz, 2H), 7.10 (t, J=7.2 Hz, 1H), 6.71-6.84 (m, 3H), 6.63 (d, J=7.5 Hz, 2H), 6.40 (d, J=8.7 Hz, 1H), 6.19 (d, J=2.7 Hz, 1H), 6.04 (br s, 1H), 5.12 (br s, 1H), 4.77 (AX, J=14.4 Hz, Δv=36.9 Hz, 2H), 3.61 (s, 3H), 3.29 (q, J=6.3 Hz, 2H), 2.94-3.13 (m, 2H), 1.97 (br s, 6H), 1.75-1.79 (m, 2H), 1.67 (br s, 1H). MS(ESI)⁺m/z 502.1 ([MNa]⁺, 100%)

### C₄DAA1106 Amide

¹H-NMR (300 MHz, CDCl₃) δ 7.27 (t, J=7.5 Hz, 2H), 7.10 (t, J=7.2 Hz, 1H), 6.92-6.99 (m, 1H), 6.65-6.84 (m, 5H), 6.56 (br s, 1H), 6.34 (d, J=8.7 H, z1H), 6.20 (d, J=3.0 Hz, 1H), 4.78 (AX, J=14.7 Hz, Δv=36.0 Hz, 2H), 3.61 (s, 3H), 3.28 (2H, m), 2.81-3.05 (m, 2H), 2.01 (s, 3H), 1.96 (s, 3H), 1.63-1.68 (m, 4H). MS(ESI)⁺ m/z 494.2 ([MH]⁺, 100%)

### C₅DAA1106 Amide

¹H-NMR (300 MHz, CDCl₃) δ 7.28 (t, J=7.5 Hz, 2H), 7.10 (t, J=7.5 Hz, 1H), 6.92-6.99 (m, 1H), 6.80-6.85 (m, 1H), 6.68-6.76 (m, 4H), 6.47 (br s, 1H), 6.39 (d, J=8.7 Hz, 1H), 6.20 (d, J=3.0 Hz, 1H), 4.81 (br s, 1H), 4.78 (AX, J=14.4 Hz, Δv=73.5 Hz, 2H), 3.61 (s, 3H), 3.28 (q, J=6.0 Hz, 2H), 2.82-3.08 (m, 2H), 1.95 (s, 3H), 1.95 (s, 3H), 1.44-1.65 (m, 7H) MS(ESI)⁺ m/z 508.2 ([MH]⁺, 100%)

### C₆DAA1106 Amide

¹H-NMR (400 MHz, CDCl₃) δ 7.28 (t, J=7.6 Hz, 2H), 7.10 (t, J=7.2 Hz, 1H), 6.92-6.97 (m, 1H), 6.80-6.84 (m, 1H), 6.70-6.75 (m, 4H), 6.40 (d, J=8.8 Hz, 1H), 6.19 (d, J=2.8 Hz, 1H), 5.90 (br s, 1H), 4.87 (br s, 1H), 4.76 (AX, J=14.4 Hz, Δv=114.0 Hz, 2H), 3.60 (s, 3H), 3.24 (q, J=6.0 Hz, 2H), 2.86-3.06 (m, 2H), 1.97 (s, 3H), 1.94 (s, 3H), 1.48-1.59 (m, 4H), 1.33-1.41 (m, 4H) MS(ESI)⁺ m/z 522.2 ([MH]⁺, 100%)

### C₇DAA1106 Amide

¹H-NMR (300 MHz, CDCl₃) δ 7.28 (t, J=7.5 Hz, 2H), 7.10 (t, J=7.2 Hz, 1H), 6.92-6.98 (m, 1H), 6.80-6.85 (m, 1H), 6.70-6.75 (m, 4H), 6.40 (d, J=8.7 Hz, 1H), 6.19 (d, J=2.7 Hz, 1H), 5.64 (br s, 1H), 4.87 (br s, 1H), 4.76 (AX, J=14.4 Hz, Δv=100.2 Hz, 2H), 3.60 (s, 3H), 3.23 (q, J=6.0 Hz, 2H), 2.86-3.03 (m, 2H), 1.96 (s, 3H), 1.94 (s, 3H), 1.45-1.60 (m, 4H), 1.31-1.36 (m, 6H). MS(ESI)⁺ m/z 536.4 ([MH]⁺, 100%)

### C₈DAA1106 Amide

¹H-NMR (300 MHz, CDCl₃) δ 7.29 (t, J=7.5 Hz, 2H), 7.10 (t, J=7.2 Hz, 1H), 6.91-6.98 (m, 1H), 6.80-6.85 (m, 1H), 6.70-6.74 (m, 4H), 6.41 (d, J=8.7 Hz, 1H), 6.20 (d, J=3.0 Hz, 1H), 5.60 (br s, 1H), 4.86 (br s, 1H), 4.76 (AX, J=14.4 Hz, Δv=113.1 Hz, 2H), 3.61 (s, 3H), 3.23 (q, J=6.0 Hz, 2H), 2.84-3.06 (m, 2H), 1.96 (s, 3H), 1.94 (s, 3H), 1.46-1.59 (m, 4H), 1.31-1.37 (m, 8H). MS(ESI)⁺ m/z 550.5 ([MH]⁺, 100%)

### C9DAA1106 Amide

¹H-NMR (300 MHz, CDCl₃) δ 7.29 (t, J=7.5 Hz, 2H), 7.10 (t, J=7.2 Hz, 1H), 6.91-6.98 (m, 1H), 6.80-6.85 (m, 1H), 6.70-6.74 (m, 4H), 6.41 (d, J=9.0 Hz, 1H), 6.20 (d, J=3.0 Hz, 1H), 5.31 (br s, 1H), 4.86 (br s, 1H), 4.76 (AX, J=14.4 Hz, Δv=110.1 Hz, 2H), 3.61 (s, 3H), 3.23 (q, J=6.0 Hz, 2H), 2.84-3.05 (m, 2H), 1.97 (s, 3H), 1.94 (s, 3H), 1.46-1.59 (m, 4H), 1.31-1.37 (m, 10H). MS(ESI)⁺ m/z 564.5 ([MH]⁺, 100%)

### IRDye™800C W-C₆DAA1106 (NIRDAA)

IRDye™800CW NHS ester (3 mg, 2.6 μmol) and C₆DAA1106 (3 mg, 6.3 μmol) were mixed in DMSO (7 mL) in a round bottom flask and stirred under argon flow for 1 hour. HPLC analysis was performed on a Varian Polaris C-18 column (250×4.6 mm) at a flow rate of 0.8 mL/min. Flow A was 0.1% TEA in water and flow B was 0.1% TEA in acetonitrile. The elution method for analytical HPLC started with a linear gradient from 100% to 70% A over 20 minutes, continued to 50% A over 5 minutes, arrived at 20% A in another 10 minutes, held at 20% A for 3 min, and finally returned to 100% A over 1 minute. The elution profile was monitored by UV absorbance at 254 and 780 nm. Product was purified by preparative HPLC using a Varian Polaris C-18 column (250×21.2 mm) at 10 mL/min. Acetonitrile in the desired fraction was removed by vacuum and the aqueous solution was loaded on an ion exchange column loaded with Amberlite IR-120 plus ion exchange resin (Sodium form). The collected solution was concentrated by vacuum rotary evapotation, frozen to −78° C. and dried under freeze-dry system. NIRDAA was collected as a dark green solid (1.2 mg, 31%). 1H NMR 500 MHz (MeOD) 7.99-7.91 (m, 3H), 7.86-7.78 (m, 6H), 7.34 (d, J=8.0 Hz, 1H), 7,27-7.23 (m, 3H), 7.17 (d, J=8.5 Hz, 1H), 7.10-7.01 (m, 3H), 6.77-6.74 (m, 1H), 6.70 (dd, J=9.0, 3.0 Hz, 1H), 6.59 (d, J=8.5 Hz, 2H), 6.42 (d, J=9.0 Hz, 1H), 6.26 (d, J=14.0 Hz, 1H), 6.20 (d, J=3.0 Hz, 1H), 6.15 (d, J=14.0 Hz, 1H), 4.96 (d, J=14.5 Hz, 1H), 4.60 (d, J=14.5 Hz, 1H), 4.15-4.10 (m, 2H), 4.08-4.05 (m, 2H), 3.52 (s, 3H), 3.43-3.39 (m, 2H), 3.14-3.11 (m, 2H), 3.04-2.93 (m, 2H), 2.89-2.86 (m, 2H), 2.82-2.72 (m, 5H), 2.17 (t, J=7.0 Hz, 2H), 2.05-2.02(m, 2H), 1.96-1.91 (m, 8H), 1.79-1.76 (m, 3H), 1.68-1.63 (m, 3H), 1.53-1.41 (m, 6H), 1.37 (d, J=4.0 Hz, 12H). MS(ESI)⁺ m/z 732.8 ([M3H]²⁺, 100%)

### Lissamine™-C₆DAA1106 (LissDAA)

A mixture of lissamine™ rhodamine B sulphonyl chloride (10 mg, 17 μmol), conjugable DAA1106 (10 mg, 20 μmol) and tri-ethylamine (15 μL) in dichloromethane (1.6 mL) was stirred under argon at room temperature for 1 hour. The reaction solution was concentrated by rotary evaporation and the crude product was purified through column chromatography (silica gel) using a 19:1 dichloromethane:methanol solution to yield LissDAA as pink solid. (Isomer I, 5.7 mg, 32%; Isomer II, 4.7 mg, 27%). 1H NMR 400 MHz (CDCl3) Isomer I; δ 8.84 (s, 1H), 7.98 (d, J=7.6 Hz, 1H), 7.30-7.24 (m, 3H), 7.19 (d, J=7.6 Hz, 1H), 7.08 (t, J=7.2 Hz, 1H), 6.93-6.90 (m, 2H), 6.78 (t, J=8.8 Hz, 3H), 6.70 (dd, J=8.4, 2.0 Hz, 1H), 6.66-6.63 (m, 3H), 6.37 (d, J=8.4 Hz, 1H), 6.19 (s, 1H), 5.61 (t, J=5.2 Hz, 1H), 4.78 (d, J=6.4 Hz, 1H), 3.59 (s, 3H), 3.56-3.45 (m, 7H), 3.10 (q, J=6.4 Hz, 2H), 3.02-2.96 (m, 1H), 2.87-2.81 (m, 1H), 2.01 (s, 2H), 1.72-1.50 (m, 12H), 1.44-1.37 (m, 4H), 1.21 (t, J=6.8 Hz, 3H). Isomer II: δ 8.72 (s, 1H), 8.36 (d, J=7.2 Hz, 1H), 7.27-7.18 (m, 5H), 7.08 (t, J=7.6 Hz, 1H), 6.95-6.89 (m, 2H), 6.87-6.85 (m, 3H), 6.79-6.76 (m, 1H), 6.71 (d, J=2.4 Hz, 2H), 6.65 (d, J=8.0 Hz, 2H), 6.20 (d, J=2.8 Hz, 1H), 6.05-6.00 (m, 1H), 4.77 (s, 2H), 3.62-3.56 (m, 10H), 3.50-3.45 (m, 1H), 3.32-3.27 (m, 1H), 3.02-2.96 (m, 1H), 2.96-2.91 (m, 3H), 2.85-2.79 (m, 2H), 1.95 (s, 3H), 1.52-1.42 (m, 4H), 1.32 (t, J=7.2 Hz, 12H), 1.22-1.19 (m, 3H), 1.15-1.11 (m, 3H). MS (ESI): 1020.4 Da [M+Na]⁺. Rf 0.39 (Isomer I), 0.32 (Isomer II) (6% methanol in dichloromethane).

### Spectroscopic Characterization

Upon preparing NIRDAA and LissDAA, absorption and emission spectra were obtained at room temperature with a Shimadzu 1700 UV-vis spectrophotometer and ISS PCI spectrofluorometer respectively. NIRDAA was found to have an absorption maximum at 778 nm and fluorescence maximum at 800 nm in methanol. The two isomers of LissDAA have similar absorption maximum (isomer I at 561 nm and isomer II at 563 nm) and same fluorescence maximum at 583 nm.

### Binding Studies

PBR protein was harvested from MA-10 cells and stocked in PBS at 10 mg/mL. The stock solution was diluted to 30 μg/100 μL for the binding study. [³H]PK11195 was used as radioligand and the specific activity of the stock solution was 73.6 Ci/mmole (˜11.8 μM). A diluted solution of 15 nM in PBS was prepared before use. For each of the molecules tested (NIRDAA, LissDAA and C₃₋₉DAA1106 amide), eight concentrations of solutions from 3×10⁻⁴M to 3×10⁻¹¹M in PBS buffer were prepared. 30 test tubes were used for the study of each molecule. 3 of them were used for total binding (100 μL 15 nM [³H]PK11195+100 μL PBS buffer+100 μL 30 μg/100 μL PBR protein), 3 test tubes were for non-specific binding (100 μL 15 nM [³H]PK11195+100 μL 15 μM PK11195+100 μL 30 μg/100 μL PBR protein) and the other 24 were triplicates for each doses of competitor ligand (100 μL, 15 nM [³H]PK11195+100 μL competitor solution+100 μL 30 μg/100 μL PBR protein). All test tubes were vortexed and then incubated at 4° C. for 90 minutes. Reactions were stopped by filtration on FG/B filters using the Brandel binding apparatus through Whatman GF/B filters (Brandel, Gaithersburg, Md.). The filters (24 positions each) were preincubated in PBS/H₂O containing 0.05% PEI for 20-30 minutes before filtration. The filters were then washed 5 times with PBS, put in scintillation vials, vortexed in scintillation fluid and counted. The 1050 and Ki (equilibrium dissociation constant) values were calculated using the PRISM program package

### Cell Imaging

C6 glioma cells were cultured in Dulbecco's modified Eagle medium (DMEM)-F12 medium (Gibco/Invitrogen) supplemented with 0.1% gentamicin sulfate (Biowhittaker). MDA-MB-231 human mammary adenocarcinoma breast cancer cells cells were cultured in closed cap flasks, with 90% Leibovitz's L-15 medium, supplemented with 2 mM L-glutamine and 10% fetal bovine serum. MDA-MB-231 or C6 cells in MaTek dishes were incubated with 1 μM NIRDAA and 1 μM LissDAA in culture media for 30 minutes. 1 nM MitoTracker Green was then added to the cell plate and incubated for another 10 minutes. Cells were rinsed and re-incubated with saline before imaging on a Nikon epifluorescence microscope equipped with Hamamatsu C4742-98 camera, Nikon Plan Apo 60x/1.40 oil objective, a mercury lamp, an ICG filter set and a Fitc filter set.

### Apoptosis Study Using Etoposide and EtoposideDAA

50,000 MDA-MB-231 human breast tumor (high PBR expressing) or human Jurkat T lymphocyte cells (low PBR expressing) cells/well were added to 96 well plates and incubated under standard tissue culture conditions (37° C., 5% CO₂) for 24 hours. Etoposide-C₆DAA1106 or etoposide were added at concentrations ranging from 100 μM to 100 pM. After three days, CellTiter-Glo luminescent cell viability assay (Promega) was added. The plates were incubated for an additional one hour and the fluorescence was counted under Xenogen IVIS imaging system. Three wells were used for control which had cells treated with viability assay without drug. Blank sampless did not have cells or drug, but had viability assay. Medium samples had cells only, without drug or viability assay.

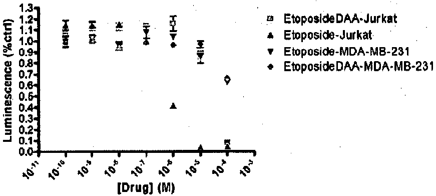

Cytotoxicity comparison between etoposide-DAA and etoposide is shown in the above table. None of control, blank or medium samples gave any significant signal. Jurkat cells began to respond to etoposide at 10⁻⁶ M, and they were effectively responding at 10⁻⁵ M. However, Jurkat cells effectively respond to etoposide-C₆DAA1106 at 10⁻⁴ M. This indicates that etoposide-C₆DAA1106 has less toxicity to normal cells than etoposide. MDA-MB-231 cells were beginning to respond to etoposide and DAA-etoposide at 10⁻⁴ M. This shows that etoposideDAA and etoposide have similar efficiency in killing high PBR expressing cancer cells

### REFERENCES

Throughout the application, various publications are cited. The publications, including those listed below, are incorporated herein by reference in their entirety.
Decaudin, D.; Castedo, M.; Nemati, F.; Beurdeley-Thomas, A.; De Pinieux, G.; Caron, A.; Pouillart, P.; Wijdenes, J.; Rouillard, D.; Kroemer, G.; Poupon, M. F. Cancer Res 2002, 62, 1388-1393.
Romeo. E.; Auta, J.; Kozikowski, A. P.; Ma, D.; Papadopoulos, V.; Puia, G.; Costa, E.; Guidotti, A. J Pharmacol Exp Ther 1992, 262, 971-978.
Kozikowski, A. P.; Ma, D.; Brewer, J.; Sun, S.; Costa, E.; Romeo, E.; Guidotti, A. Journal of Medicinal Chemistry 1993, 36, 2908-2920.
Culty, M.; Silver, P.; Nakazato, A.; Gazouli, M.; Li, H.; Muramatsu, M.; Okuyama, S.; Papadopoulos, V. Drug Develop Res 2001, 52, 475-484.
Zhang, M. R.; Maeda, J.; Furutsuka, K.; Yoshida, Y.; Ogawa, M.; Suhara, T.; Suzuki, K. Bioorg Med Chem Lett 2003, 13, 201-204.
Maeda, J.; Suhara, T.; Zhang, M. R.; Okauchi, T.; Yasuno, F.; Ikoma, Y.; Inaji, M.; Nagai, Y.; Takano, A.; Obayashi, S.; Suzuki, K. Synapse 2004, 52, 283-291.
Chaki, S.; Funakoshi, T.; Yoshikawa., R.; Okuyama, S.; Okubo, T.; Nakazato, A.; Nagamine, M.; Tomisawa, K. Eur J Pharmacol 1999, 371, 197-204.
Manning, H. C.; Goebel, T.; Marx, J. N.; Bomhop, D. J. Org Lett 2002, 4, 1075-1078.
Manning, H. C.; Smith, S. M.; Sexton, M.; Haviland, S.; Bai, M. F.; Cederquist, K.; Stella, N.; Bornhop, D. J. Rioconjugate Chem 2006, 17, 735-740.
Okubo, T.; Yoshikawa, R.; Chaki, S.; Okuyama, S.; Nakazato, A. Bioorgan Med Chem 2004, 12, 423-438.
Zhang, M. R.; Kida, T.; Noguchi, J.; Furutsuka, K.; Maeda, J.; Suhara, T.; Suzuki, K. Nucl Med Biol 2003, 30, 513-519.
Hasinoff B B, Chee G L, Day B W, Avor K S, Bamabe N, Thampatty P, et al. Synthesis and biological activity of a photoaffinity etoposide probe. Bioorganic & Medicinal Chemistry 2001; 9:1765-1771.
Ishiguro K. Taft W C, Delorenzo R J, Sartorelli A C. The Role of Benzodiazepine Receptors in the Induction of Differentiation of Hl-60 Leukemia-Cells by Benzodiazepines and Purines. Journal of Cellular Physiology 1987; 131:226-234.
Alexander B E E, Roller E, Klotz U. Characterization of Peripheral-Type Benzodiazepine Binding-Sites on Human-Lymphocytes and Lymphoma Cell-Lines and Their Role in Cell-Growth. Biochemical Pharmacology 1992; 44:269-274.
Culty M, Silver P, Nakazato A, Gazouli M, Li H, Muramatsu M, et al. Peripheral benzodiazepine receptor binding properties and effects on steroid synthesis of two new phenoxyphenyl-acetamide derivatives, DAA1097 and DAA1106. Drug Development Research 2001; 52:475-484.
Okaro A C, Fennell D A, Corbo M, Davidson B R, Cotter F E. Pk11195, a mitochondrial benzodiazepine receptor antagonist, reduces apoptosis threshold in Bcl-X-L and Mcl-1 expressing human cholangiocarcinoma cells. Gut 2002; 51:556-561.
Hirsch T, Decaudin D, Susin S A, Marchetti P, Larochette N, Resche-Rigon M, et al. PK11195, a ligand of the mitochondrial benzodiazepine receptor, facilitates the induction of apoptosis and reverses Bcl-2-mediated cytoprotection. Experimental Cell Research 1998; 241:426-434.

The invention thus being described it will be obvious that the same can be changed in many ways.

1-19. (canceled)

20. A compound of the following formula: 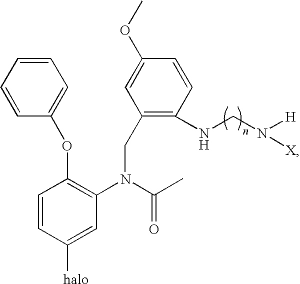 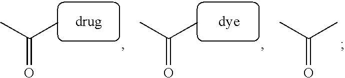
wherein n is 1-10, and X is H, and stereoisomers and conjugable analogs thereof.

21. A compound of claim 20, of the following formula: 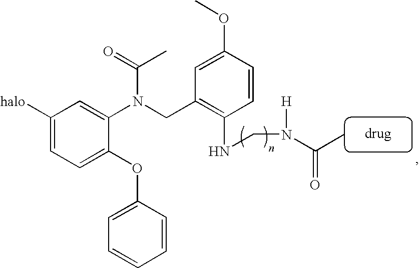
and stereoisomers and conjugable analogs thereof.

22. A compound of claim 20, of the following formula: 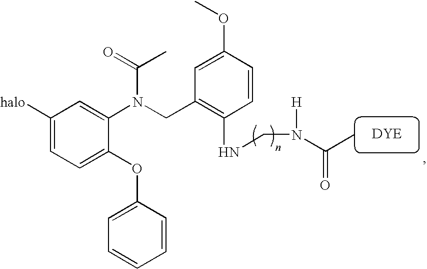
and stereoisomers and conjugable analogs thereof.

23. A compound of claim 22, of the following formula: 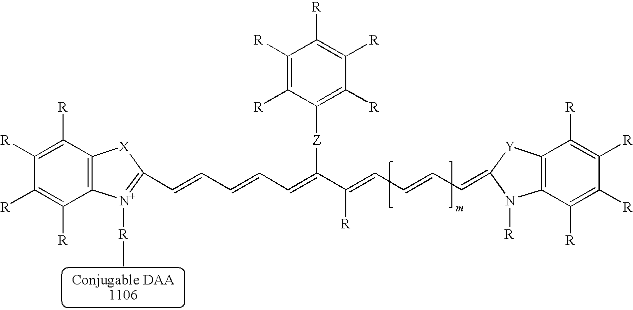
wherein Z is O, S, or NR₃₅ wherein R₃₅ is H or alkyl;
R₁-R₅ are each independently H, alkyl, halo, carboxyl, amino, or SO₃⁻Cat⁺, wherein Cat⁺ is a cation and at least one of R₁-R₅ is SO₃⁻Cat;
R₆ and R₇ are each H, alkyl, or optionally, together with the 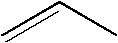
group to which they are bonded, form a ring;
m and n are each independently integers from 0 to 5;
X and Y are each independently O, S, Se, or CR₁₉ R₂₀, wherein R₁₉ and R₂₀ are each independently alkyl, or optionally form a ring together with the carbon atom to which they are bonded;
R₈ is a member selected from the group consisting of alkyl, (CH₂)ᵣR₂₅, (CH₂)ᵣR₁₈, and R₃₀—B, wherein r is an integer from 1 to 50, and R₂₅ is a functional group that does not directly react with a carboxyl, hydroxyl, amino, or a thiol group, and R₁₈ is a functional group that can react with a carboxyl, hydroxyl, amino, or thiol group, or wherein said R₁₈ group is inactivated; and
R₉-R₁₂ and R₁₄-R₁₇ are each independently H, alkyl, halo, amino, sulfonato, R₂₁COOH, R₂₁OR₂₂, R₂₁SR₂₂, or R₂₁COOR₂₂ wherein R₂₁ is a bond or alkylene and R₂₂ is alkyl, or optionally R₁₁ and R₁₂ together with the atoms to which they are bonded form an aromatic ring, or optionally R₁₆ and R₁₇ together with the atoms to which they are bonded form an aromatic ring;
B is a biomolecule; and
R₃₀ is (CH₂)ᵣL; wherein r is an integer from 1 to 50, and L is a linking group.

24. A compound of claim 20, of the following formula: 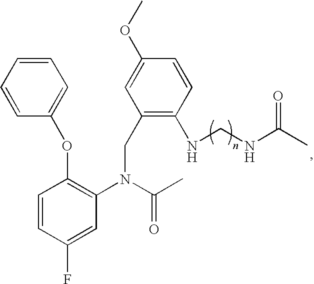
and stereoisomers and conjugable analogs thereof.

25. A compound of claim 21, wherein drug is an etopiside compound.

26. A compound of claim 25, of the following formula: 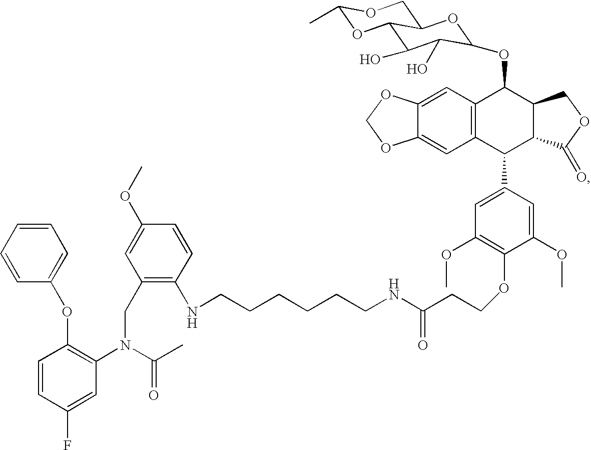
and stereoisomers and conjugable analogs thereof.

27. A method of imaging a molecular event in a sample, comprising:
(a) administering to said sample a probe having an affinity for a target, the probe comprising a compound of the following formula: 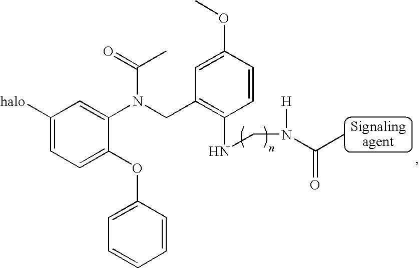
wherein n is an integer from 1 to 10; and
(b) detecting a signal from said probe.

28. The method of claim 27, wherein the signaling agent is a near infrared signaling agent.

29. The method of claim 27, further comprising the step of analyzing a disease state.

30. The method of claim 27, wherein the signaling agent is a dye.

31. A method of treating cancer and unwanted proliferation of cells in a patient, comprising administering to said patient a compound of the following formula: 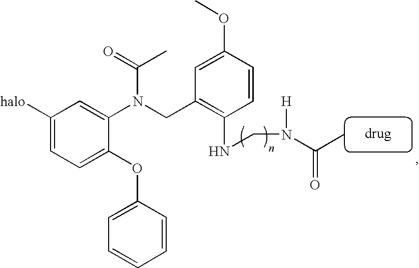
and/or a stereoisomer and/or an analog thereof.

32. The method of claim 31, wherein the drug is a topoisomerase inhibitor.

33. The method of claim 32, wherein the topoisomerase inhibitor is selected from the group consisting of adriamycin, amsacrine, camptothecin, daunorubicin, dactinomycin, doxorubicin, eniposide, epirubicin, etoposide, idarubicin, mitoxantrone, teniposide, and topotecan.

34. The method of claim 31, wherein the drug is etoposide.

35. The method of claim 31, used in the treatment of a proliferative disorder selected from the group consisting of a pancreatic cancer, renal cell cancer, Kaposi's sarcoma, chronic leukemia, chronic lymphocytic leukemia, breast cancer, sarcoma, ovarian carcinoma, rectal cancer, throat cancer, melanoma, colon cancer, bladder cancer, lymphoma, mesothelioma mastocytoma, lung cancer, liver cancers, mammary adenocarcinoma, pharyngeal squamous cell carcinoma, gastrointestinal cancer, stomach cancer, myeloma, prostate cancer, B-cell malignancies or metastatic cancers.

36. The method of claim 31, used to inhibit growth of a tumor cell selected from the group consisting of a pancreatic tumor cell, a lung tumor cell, a prostate tumor cell, a breast tumor cell, a colon tumor cell, a liver tumor cell, a brain tumor cell, a kidney tumor cell, a skin tumor cell and an ovarian tumor cell.

37. The method of claim 31, used to inhibit growth of a tumor cell selected from the group consisting of a squamous cell carcinoma, a non-squamous cell carcinoma, a glioblastoma, a sarcoma, an adenocarcinoma, a myeloma, a melanoma, a papilloma, a neuroblastoma and a leukemia cell.

38. The method of claim 11, wherein the compound is of the following formula: [chemical structure]
